(19) United States
(12) Patent Application Publication
(10) Pub. No.: US 2008/0206164 A1
(43) Pub. Date: Aug. 28, 2008

(54) Cosmetic composition comprising at least one cationic polyurethane and at least one vinyl pyrrolidone homo- or copolymer, and method of styling therewith

(76) Inventors: Isabelle Rollat-Corvol, Paris (FR); Pascale Cothias, Montigny le Bretonneux (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/007,626

(22) Filed: Jan. 14, 2008

Related U.S. Application Data
(60) Provisional application No. 60/903,517, filed on Feb. 27, 2007.

(30) Foreign Application Priority Data
Jan. 12, 2007 (FR) 0752654

(51) Int. Cl. A61K 8/87; A61K 8/04; A61K 8/72; A61Q 5/06

(52) U.S. Cl. 424/47; 424/70.15

(57) ABSTRACT

The present disclosure relates to a cosmetic composition comprising, in a cosmetically acceptable aqueous medium:
(i) at least one cationic polyurethane comprising at least one non ionic unit derived from an olefinic homopolymer and/or copolymer, and
(ii) at least one vinyl pyrrolidone homopolymer or copolymer.

This application claims benefit of U.S. Provisional Application No. 60/903,517, filed Feb. 27, 2007, the contents of which are incorporated herein by reference. This application also claims benefit of priority under 35 U.S.C. § 119 to French Patent Application No. FR 0752654, filed Jan. 12, 2007, the contents of which are also incorporated herein by reference.

The present disclosure relates to new cosmetic compositions, for instance hairstyling compositions, comprising the combination of at least one cationic polyurethane comprising non ionic units derived from at least one olefinic homo- and/or copolymer, and at least one vinyl pyrrolidone homo- or copolymer.

The use of elastic cationic polyurethanes in cosmetic compositions, such as hairstyling compositions is known.

For instance, French Patent Application No. FR 2 815 350 describes elastic cationic polyurethanes and their use for formulating hair sprays and hair styling compositions to enhance hair suppleness, i.e., making it possible to hold hair styles in a more natural way as compared to that obtained with usual fixing polymers.

French Patent Application No. FR 2 833 960 describes cosmetic hairstyling compositions, including rinse-off hair compositions, such as styling shampoos, comprising a self-adhering cationic or amphoteric polyurethane.

However, the present inventors have found that using a cationic polyurethane comprising units derived from an olefinic homo- and/or copolymer in a hair styling composition may provide an excellent hold over time, but may also lead to cosmetically poor properties and, further, may be difficult to remove with shampoo.

Surprisingly and unexpectedly, the present inventors have discovered that combining at least one vinyl pyrrolidone homo- or copolymer with these cationic polyurethanes comprising units derived from an olefinic homo- and/or copolymer makes it possible to formulate cosmetic hairstyling compositions that result in good cosmetic properties, while also resulting in improved fixing of the hair and improved ability to keep the hair in place over time.

Thus, one aspect of the present disclosure is a cosmetic composition, for example, a hair styling composition, comprising, in a cosmetically acceptable medium, at least one cationic polyurethane comprising at least one unit derived from an olefinic homo- and/or copolymer and at least one vinyl pyrrolidone homo- or copolymer.

Another aspect of the present disclosure is such a composition further comprising a gas propellant and being in the form of an aerosol.

A further aspect of the present disclosure is a hairstyling method comprising applying onto the hair the composition as disclosed herein, then optionally styling and drying the hair.

According to the present disclosure, the cosmetic composition comprises, in a cosmetically acceptable aqueous medium:

(i) at least one cationic polyurethane comprising at least one non ionic unit derived from an olefinic homopolymer and/or copolymer, and

(ii) at least one vinyl pyrrolidone homo- or copolymer.

### Cationic Polyurethanes

The at least one cationic polyurethane comprising at least one non ionic unit, derived from an olefinic homopolymer and/or copolymer represents the first component of the compositions of the present disclosure.

The cationic polyurethanes that may be suitably used in the present disclosure in at least one embodiment include but are not limited to:

(a) cationic units derived from at least one compound, such as a tertiary or a quaternary amine, comprising at least two labile hydrogen-containing reactive functions,

(b) non ionic units derived from non ionic polymers carrying labile hydrogen-containing reactive functions at their ends, wherein at least one of the (b) units, for example, at least 50% by weight of the (b) units and further, for example, all the (b) units, is chosen from at least one (b1) unit(s) derived from an olefinic homo- or copolymer carrying labile hydrogen-containing reactive functions at their ends, and

(c) units derived from at least one diisocyanate.

As used herein, “cationic unit” is understood to mean any unit that, either due to its own chemical nature, or because of its environment and/or the pH value by which it is surrounded, is in a cationic form.

As used herein, “labile hydrogen-containing reactive functions” means functions that are able, after the departure of a hydrogen atom, to form covalent bonds with the isocyanate functions of the compounds forming the (c) units. Suitable examples of such functions include but are not limited to: hydroxyl, primary amine (—NH₂) or secondary amine (—NHR), or thiol (—SH) groups.

Polycondensation of compounds carrying these labile hydrogen-containing reactive functions with diisocyanates results in polyurethanes, polyureas or polythiourethanes, depending on the nature of the labile hydrogen-carrying reactive functions (—OH, —NH₂, —NHR or —SH), respectively. All these polymers can be encompassed in the present application under the term “polyurethane”, for simplification purposes. According to at least one embodiment, the polymers of the present disclosure are authentic polyurethanes.

When the tertiary or quaternary amines forming the (a) units carry more than two labile hydrogen-containing functions, the resulting polyurethanes may have a branched structure.

According to at least one embodiment of the polyurethane according to the present disclosure, the tertiary or quaternary amines forming the cationic (a) units comprise only two labile hydrogen-containing reactive functions and consequently the polyurethanes resulting from the polycondensation have a substantially linear structure.

It is also possible to use a mixture of difunctional amines comprising a small proportion of amines carrying more than two labile hydrogen-containing reactive functions.

In at least one embodiment, the tertiary or quaternary amines forming the cationic (a) units may be chosen from compounds corresponding to one or more of the following formulas:

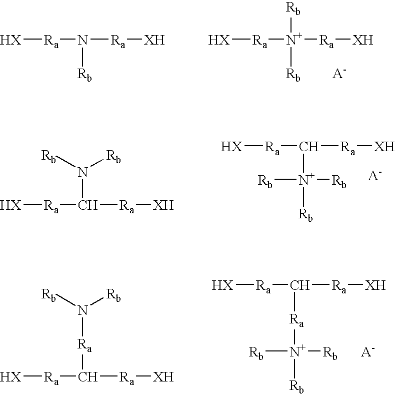

wherein:

each Rₐ is independently chosen from linear or branched C₁₋₆ alkylene, C₃₋₆ cycloalkylene and arylene groups, where all of them may be substituted with at least one halogen atom and comprise at least one heteroatom chosen from O, N, P and S;

each R_{b} is independently chosen from C₁₋₆ alkyl, C₃₋₆ cycloalkyl, and aryl groups, where all of them may be substituted with at least one halogen atom and comprise at least one heteroatom chosen from O, N, P and S;

each X is independently chosen from oxygen and sulfur atoms and from NH and NR_{c} groups, where R_{c} is a C₁₋₆ alkyl group; and

A⁻ is a physiologically acceptable counter-ion.

According to at least one embodiment, N-methyldiethanol amine and N-tert-butyldiethanol amine are tertiary amines that can be used for producing said cationic polyurethanes.

Tertiary and quaternary amines forming the cationic (a) units of the polyurethanes of the present disclosure may also be tertiary and/or quaternary amine function-containing polymers, carrying labile hydrogen-containing reactive functions at their ends. Weight average molecular weight of such tertiary and/or quaternary amine function-containing polymers can range, in at least one embodiment, from 400 to 10,000.

As suitable examples of such amine function-containing polymers, polyesters resulting from the polycondensation of N-methyldiethanol amine and adipic acid may be mentioned.

When the amines forming the cationic (a) units are tertiary amine function compounds, all or part of these amine functions are neutralized with a suitable neutralizing agent chosen from physiologically acceptable organic or mineral acids. Hydrochloric acid or acetic acid may be used, for example.

According to at least one embodiment, the second type of unit that may form the polyurethanes of the present disclosure includes macromolecular units, called (b) units, derived from non ionic polymers carrying labile hydrogen-containing reactive functions at their ends and according to at least one embodiment, having a glass transition temperature (Tg) lower than 10° C., as measured by differential enthalpy analysis.

According to the present disclosure, at least one of these (b1) units can be derived from an olefinic homo- or copolymer.

The polyurethane viscoelastic properties can be used when (b) units are derived from polymers having a glass transition temperature lower than 0° C., for example lower than −10° C.

These polymers may have a weight average molecular weight ranging from 400 and 10,000 and further, for example, from 1000 to 5000.

Non ionic polymers that can form (b2) non ionic units that differ from (b1) non ionic units derived from olefinic homo- and copolymers, may be chosen from polyethers, polyesters, polysiloxanes, polycarbonates and fluorinated polymers, for example.

According to at least one embodiment, polymers that can form said (b) non ionic units are chosen from olefinic homo- and copolymers.

Olefinic polymers having labile hydrogen-containing reactive groups on their terminal ends, to be suitably used in the present disclosure, include, but are not limited to, ethylene, propylene, 1-butylene, 2-butylene, isobutylene, 1,2-butadiene, 1,4-butadiene and isoprene random or block homopolymers and copolymers.

Butadiene and isoprene homo- and copolymers may be partially or fully hydrogenated.

In at least one embodiment, polymers that may be used are copolymers of ethylene/butylene, polybutadienes and hydrogenated polybutadienes carrying on their terminal ends labile hydrogen-containing reactive groups, for example, hydroxyl groups. According to at least one embodiment, these polymers are 1,2- and/or 1,4-polybutadienes.

Such polymers are commercially available, for example, under the trade name KRATON® L, for example KRATON® L 2203 (hydrogenated polybutadiene diol) from the KRATON polymers company, KRASOL LBH® and LBHP®, such as KRASOL LBHP® 2000 (polybutadiene diol) from the SARTOMER company and GI® 3000 (copolymer of ethylene and butylene) from the NISSO CHEMICAL company.

The diisocyanates forming the (c) units may include aliphatic, alicyclic or aromatic diisocyanates.

For example, diisocyanates can be chosen from methylenediphenyl diisocyanate, methylenecyclohexane diisocyanate, isophorone diisocyanate, toluene diisocyanate, naphthalene diisocyanate, butane diisocyanate and hexyl diisocyanate. These diisocyanates may of course be used alone or as a mixture of two or more diisocyanates. According to at least one embodiment, the diisocyanate is isophorone diisocyanate.

As previously mentioned, cationic polyurethanes of the present disclosure may contain, in addition to (a), (b1) and (c) units, a certain content of (b2) units derived from monomeric, non-ionic compounds, comprising at least two labile hydrogen functions, that differ from the compounds leading to the (b1) units.

These (b2) units may be derived from C₁-C₁₂ diols, for example from neopentyl glycol, hexaethylene glycol, 1,2-ethanediol, 1,2-propanediol and 1,3-propanediol or from C₁-C₆ aminoalcohols, for example, from aminoethanol.

The cationic polyurethanes of the present disclosure may be elastic.

According to at least one embodiment of the present disclosure, the cationic polyurethane does not comprise any further unit in addition to (a), (b) and (c) units. For instance, the polyurethane (A) described in the examples below is a polyurethane corresponding to such definition.

In an alternative embodiment, the cationic polyurethane comprises further units, in addition to (a), (b) and (c) units. For instance, the polyurethane (B) described in the examples below is a polyurethane corresponding to such definition.

A physical parameter characterizing the viscoelastic properties of the above cationic polyurethanes is their tensile recovery. Such recovery may be determined by a tensile creep test by rapidly stretching a specimen to a predetermined degree of elongation, then in releasing the stress, and lastly in measuring the specimen length.

The creep test used to characterize the cationic polyurethanes with elastic character of the present disclosure is performed as follows:

The specimen used is a film of polyurethane 500±50 mm-thick, cut into 80 mm×15 mm strips. This copolymer film is obtained by drying at a temperature of 22±2° C. under a 50±5% relative humidity, a 3% by weight solution or dispersion of said polyurethane in water and/or in ethanol.

Each strip is fixed between two jaws, spaced apart from each other by 50±1 mm, and is stretched at a speed of 20 mm/minute (under the hereabove mentioned temperature and relative humidity conditions) up to a 50% elongation (εₘₐₓ), i.e., until a strip is obtained, wherein the size corresponds to 1.5 times its initial length. The stress is then released by setting a return speed equal to the tensile speed, i.e., 20 mm/minute, and the specimen elongation is then measured (as expressed in % relative to the initial length) immediately once it has returned to a zero load (εᵢ).

The instantaneous recovery (Rᵢ) is calculated using the following equation:

Rᵢ(%)=((εₘₐₓ−εᵢ)/εₘₐₓ)×100

In at least one embodiment, the elastic cationic polyurethanes of the present disclosure have an instantaneous recovery (Rᵢ), such as measured in the above stated conditions, ranging from 5% to 95%, such as from 20% to 90%, and according to a further embodiment, ranging from 35% to 85%.

The glass transition temperature (Tg) of the non ionic polymers forming the (b) units and of the cationic polyurethanes of the present disclosure is measured by means of a differential enthalpy analysis (DSC, differential scanning calorimetry) according to ASTM D3418-97 standard.

In at least one embodiment, elastic cationic polyurethanes of the present disclosure have at least two glass transition temperatures, at least one of which is lower than 10° C., such as lower than 0° C., for example lower than −10° C., and the other one of which is at least higher than or equal to the room temperature (20° C.).

The instantaneous recovery and therefore the viscoelastic properties of the polyurethanes of the present disclosure depend on the contents of the various (a), (b1), (b2) and (c) monomer units.

In at least one embodiment, the (a) unit content is sufficient to provide the polymers with the positive charge responsible for their good affinity for keratinic substrates, and the (b) units are present in an amount by weight sufficient for the polyurethanes to have, for instance, at least one glass transition temperature lower than 10° C. and not to form brittle films.

According to at least one embodiment, the (a) units are present in an amount ranging from 0.1 to 90%, for instance from 1 to 30%, for example, from 5 to 25% and further, for example, from 5 to 10% by weight, the (b1) units are present in an amount ranging from 10 to 99.9%, for instance from 20 to 99% and further, for example, from 30 to 85% by weight, and the (b2) units are present in an amount ranging from 0 to 50% by weight, for example from 0 to 30% by weight, relative to the total weight of the polyurethane units. According to at least one embodiment, the polyurethanes of the present disclosure do not comprise any (b2) units.

In at least one embodiment, the (c) units are present in an amount ranging from 1 to 60%, such as from 5 to 50%, for example from 10 to 40% of the polyurethane unit total weight.

In at least one embodiment, the (c) units are present in a substantially stoichiometric amount as compared to the sum of (a) and (b) units. Obtaining polyurethanes with high molecular weights requires a number of isocyanate functions almost identical to the number of labile hydrogen functions. The person skilled in the art will be able to choose an optional molar excess of the one function or the other, to adjust the molecular weight to the expected value.

The amount of polyurethane present in a cosmetic composition of the present disclosure depends on the composition type and the required properties, and may vary within a very broad range, ranging from 0.01 to 40% by weight, for example ranging from 0.05 to 20%, and further, ranging from 0.1 to 10% by weight, relative to the final cosmetic composition.

### Vinyl Pyrrolidone Homopolymer or Copolymer

The second main component of the compositions of the present disclosure is a vinyl pyrrolidone homopolymer or copolymer.

Amongst homopolymers, polyvinyl pyrrolidones (PVP) having various molecular weights may be mentioned, by way of non-limiting example.

Examples of suitable copolymers include but are not limited to copolymers of polyvinyl pyrrolidone and vinyl acetate (PVP/VA), of polyvinyl pyrrolidone and butene; of polyvinyl pyrrolidone/decene, of polyvinyl pyrrolidone/hexadecene, of polyvinyl pyrrolidone/dimethyl aminoethyl methacrylate (PVP/DMAEMA), of polyvinyl pyrrolidone/dimethylaminopropyl(meth)acrylamide, of polyvinyl pyrrolidone/(meth)acrylic acid/lauryl methacrylate, of polyvinyl pyrrolidone/vinyl acetate/itaconic acid, of polyvinyl pyrrolidone/vinyl acetate/vinyl propionate, and of polyvinyl pyrrolidone/vinyl caprolactame/dimethylaminopropyl acrylamide/acrylic acid or ester.

According to at least one embodiment, polyvinyl pyrrolidones (PVP) and copolymers of vinyl pyrrolidone and vinyl acetate (PVP/VA) are homo- et copolymers that can be used in the present disclosure.

These copolymers are available commercially, for example, under the trade name LUVISKOL® (PVP and PVP/VA) from the BASF company and under the trade names PVP-K or PVP/VA from the ISP company.

For instance, the homo- or copolymer of vinyl pyrrolidone included in the composition is present in an amount ranging from 0.01 to 20%, for example from 0.1 to 10% and according to at least one embodiment, ranging from 0.5 to 5% by weight, relative to the total weight of the composition.

### Cosmetically Acceptable Additives and Solvents

The cosmetically acceptable aqueous medium may comprise various additives and solvents that are commonly used in the cosmetic field such as surfactants, gelling agents and/or thickeners, silicones, organic solvents, fragrances, mineral, vegetable and/or synthetic oils or waxes, fatty acid esters, pigments and dyes, mineral or organic particles, pH stabilizing agents, preserving agents, and UV absorbers.

As disclosed herein, the surfactants that may be used in the composition include but are not limited to anionic, non ionic, amphoteric or cationic surfactants, and mixtures thereof.

Non-limiting examples of suitable anionic surfactants that may be used either alone or in combination in the context of the present disclosure include salts, for example alkaline metal salts such as sodium salts, ammonium salts, amine salts, aminoalcohol salts or alkaline-earth metal salts, for example, magnesium salts, of the following compounds: alkyl sulfates, alkyl ethersulfates, alkyl amidoethersulfates, alkylaryl polyethersulfates, monoglyceride sulfates; alkyl sulfonates, alkyl amidesulfonates, alkyl-aryl sulfonates, α-olefin sulfonates, paraffin sulfonates; alkyl sulfosuccinates, alkyl ethersulfosuccinates, alkylamide sulfosuccinates; alkyl sulfoacetates; acyl sarconisates; and acylglutamates, wherein the alkyl and acyl groups of all these compounds may comprise from 6 to 24 carbon atoms, for example, wherein the aryl group may correspond to a phenyl or benzyl group, for instance.

Polyglycoside carboxylic acid and C₆-C₂₄ alkyl esters may also be used in the context of the present disclosure, such as alkyl glucoside citrates, alkyl polyglycoside tartrates and alkyl polyglycoside sulfosuccinates; as well as alkyl sulfosuccinamates, acyl isethionates and N-acyl taurates, wherein the alkyl or acyl group of these compounds may comprise from 12 to 20 carbon atoms, for example. Further non-limiting mention may be made of suitable anionic surfactants such as acyl lactylates the acyl group of which comprises from 8 to 20 carbon atoms, for example.

Moreover, alkyl-D-galactoside uronic acids and salts thereof may also be mentioned, as well as polyoxyalkylene (C₆-C₂₄)alkylether carboxylic acids, polyoxyalkylene (C₆-C₂₄)alkyl(C₆-C₂₄)arylether carboxylic acids, polyoxyalkylene (C₆-C₂₄)alkylamidoether carboxylic acids and salts thereof, for example those comprising from 2 to 50 ethylene oxide groups, and mixtures thereof.

Amongst the above mentioned anionic surfactants, according to at least one embodiment of the present disclosure, (C₆-C₂₄)alkyl sulfates, (C₆-C₂₄)alkyl ethersulfates, (C₆-C₂₄)alkyl ethercarboxylates and mixtures thereof, for example ammonium lauryl sulfate, sodium lauryl sulfate, magnesium lauryl sulfate, sodium lauryl ethersulfate, ammonium lauryl ethersulfate and magnesium lauryl ethersulfate may be used.

The composition of the present disclosure may comprise anionic surfactants in an amount ranging from 0.5 to 60% by weight, for example, ranging from 5 to 20% by weight, relative to the total weight of the composition.

Non ionic surfactants that can be used in the context of the present disclosure include, but are not limited to compounds that are well known by those of skill in the art (for a review thereof, see, e.g., “Handbook of Surfactants” M. R. PORTER, Blackie & Son Editor (Glasgow and London), 1991, pp 116-178). For instance, they may be chosen from alcohols, alpha-diols, (C₁-C₂₀)alkyl phenols or polyethoxylated, polypropoxylated or polyglycerolated fatty acids, having a fatty chain comprising, for example, 8 to 18 carbon atoms, where the number of ethylene oxide or propylene oxide groups may range from 2 to 50 and the number of glycerol groups may range from 2 to 30. Non-limiting mention can also be made of copolymers of ethylene oxide and propylene oxide, condensation products of ethylene oxide and propylene oxide on fatty alcohols; polyethoxylated fatty amides having, for example, from 2 to 30 moles of ethylene oxide; polyglycerolated fatty amides comprising on average from 1 to 5 glycerol groups and further, for example from 1.5 to 4; polyethoxylated fatty amines having, for example, from 2 to 30 moles of ethylene oxide; sorbitane fatty acid esters ethoxylated with from 2 to 30 moles of ethylene oxide; sucrose fatty acid esters, polyethylene glycol fatty acid esters, (C₆-C₂₄)alkyl polyglucosides, (C₆-C₂₄)N-alkyl glucamine derivatives, amine oxides such as (C₁₀-C₁₄)alkyl amine oxides or (C₁₀-C₁₄)N-acyl aminopropylmorpholine oxides; and mixtures thereof.

Amongst the previously mentioned non ionic surfactants, the (C₆-C₂₄)alkyl polyglycosides are used in at least one embodiment, for example decyl polyglucoside may be used.

Amphoteric surfactants that can be suitably used in the present disclosure include but are not limited to secondary or tertiary aliphatic amine derivatives, wherein the aliphatic group is a linear or a branched chain comprising from 8 to 22 carbon atoms and containing, at least one hydrosolubilizing anionic group such as, for example, a carboxylate, sulfonate, sulfate, phosphate or phosphonate group; (C₈-C₂₀)alkyl betaines, sulfobetaines, (C₈-C₂₀)alkyl (C₆-C₈)amidoalkyl betaines and (C₈-C₂₀)alkyl (C₆-C₈)amidoalkyl sulfobetaines, as well as mixtures thereof, may also be mentioned.

Amongst the amine derivatives, products marketed under the trade name MIRANOL® may be mentioned, such as those described in U.S. Pat. Nos. 2,528,378 and 2,781,354 and classified in the CTFA dictionary, Third Edition, 1982, under the names amphocarboxyglycinate and amphocarboxypropionate having respectively the following structures (1) and (2):

R₂—CONHCH₂CH₂—N⁺(R₃)(R₄)(CH₂COO⁻) (1)

wherein:

R₂ is chosen from an alkyl group derived from a R₂—COOH acid present in hydrolyzed coconut oil, and from heptyl, nonyl and undecyl groups,

R₃ is a beta-hydroxyethyl group, and

R₄ is a carboxymethyl group;

and

R₂—CONHCH₂CH₂—N(B)(C) (2)

wherein:

B is —CH₂CH₂OX′,

C is —(CH₂)_{z}—Y′, with z=1 or 2,

X′ is chosen from a —CH₂CH₂—COOH group and a hydrogen atom,

Y′ is chosen from —COOH, and a —CH₂—CHOH—SO₃H group,

R₂ is chosen from the alkyl group of a R₂—COOH acid present in hydrolyzed coconut oil or linseed oil, and from an alkyl group, such as a C₁₇ alkyl group and its iso-form, an unsaturated C₁₇ group.

These compounds are classified in the CTFA dictionary, 5th Edition, 1993, under the names disodium cocoamphodiacetate, disodium lauroamphodiacetate, disodium capryl amphodiacetate capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

The cocoamphodiacetate marketed under the trade name MIRANOL® C2M concentrated by the RHODIA company is a suitable example thereof.

Amongst suitable amphoteric surfactants, non-limiting mention can be made of (C₈-C₂₀)alkyl betaines, such as coco betaine, (C₈-C₂₀)alkyl (C₆-C₈)amidoalkyl betaines such as cocamido betaine, alkyl amphodiacetates such as disodium cocoamphodiacetate, and mixtures thereof.

Moreover, the composition of the present disclosure may further comprise at least one cationic surfactant that is well known by those of skill in the art, including but not limited to the salts of primary, secondary or tertiary fatty amines, optionally polyoxyalkylenated, quaternary ammonium salts such as tetraalkylammonium, alkylamidoalkyl trialkylammonium, trialkylbenzylammonium, trialkylhydroxyalkylammonium or alkylpyridinium chlorides or bromides, imidazoline derivatives; or amine oxides of cationic nature.

The previously described non ionic, amphoteric and cationic surfactants may be used either alone or in combination, and they can be present in an amount ranging from 0.1 to 30% by weight, for example from 0.5 to 25% and further, for example, from 1 to 20% by weight, relative to the total weight of the composition.

Gelling agents and/or thickeners that may be suitably used in the compositions of the present disclosure are well known in the art and may be chosen from carboxyvinyl polymers and copolymers, (alkyl)acrylic polymers and copolymers, (alkyl)acrylamide polymers and copolymers, poly(oxyalkylene)glycols, poly(oxyalkylene)glycol esters, alginates, biosaccharides, polysaccharides such as cellulose and starch derivatives, naturally occurring gums such as xanthan gum, guar gum, locust bean gum, scleroglucans, chitin and chitosan derivatives, carrageenans, clays, and mixtures thereof, for example.

Non-limiting examples of gelling agents, for instance in an aqueous phase, include SEPIGEL® 305 marketed by the SEPPIC company, FUCOGEL® 1000 PP marketed by the SOLABIA company, SYNTHALEN® K marketed by the 3VSA company, LUVISKOL® VA 64 P marketed by the BASF company, HOSTACERIN® AMPS marketed by the CLARIANT company, PEMULEN® TR1 marketed by the GOODRICH company, LUBRAGEL® MS marketed by the GUARDIAN company, SATIAGEL® KSO marketed by DEGUSSA and KELTROL® marketed by the KELCO company.

The gelling agent may be present in an amount ranging from 0.05 to 15%, for example from 0.5 to 10% by weight, relative to the total weight of the composition.

The silicones that may be used as additives in the cosmetic compositions of the present disclosure are volatile or non volatiles, cyclic, linear or branched silicones, optionally modified with organic groups, and having a viscosity ranging from 5×10⁻⁶ to 2.5 m²/s at 25° C., for example from 1×10⁻⁵ to 1 m²/s.

The silicones that may be used according to the present disclosure may be soluble or insoluble in the composition and for instance may be polyorganosiloxanes insoluble in the composition of the present disclosure. They may be in the form of oils, waxes, resins or gums.

Organopolysiloxanes are defined in more detail by Walter NOLL in “Chemistry and Technology of Silicones” (1968) Academie Press. Those useful herein may be volatile or not.

When they are volatile, silicones can be chosen from those having a boiling point that ranges from 60° C. to 260° C., and further, for example from the following:

(i) cyclic silicones comprising from 3 to 7, such as 4 or 5 silicon atoms. Suitable examples thereof include octamethyl cyclotetrasiloxane marketed under the trade name “VOLATILE SILICONE® 7207” by UNION CARBIDE or “SILBIONE® 70045 V 2” by RHODIA, decamethyl cyclopentasiloxane marketed under the trade name “VOLATILE SILICONE® 7158” by UNION CARBIDE, “SILBIONE® 70045 V 5” by RHODIA, as well as mixtures thereof.

Cyclocopolymers of the dimethyl siloxane and methylalkyl siloxane type may also be mentioned, such as “SILICONE VOLATILE® FZ 3109” marketed by the UNION CARBIDE company, having the following formula:

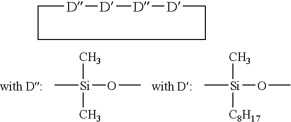

Mixtures of cyclic silicones with organic compounds derived from silicon may also be mentioned, such as a octamethyl cyclotetrasiloxane and tetratrimethylsilyl pentaerythritol mixture (50:50) and a octamethyl cyclotetrasiloxane and oxy-1,1′-(hexa-2,2,2′,2′,3,3′-trimethylsilyloxy)bis-neopentane mixture;

(ii) linear volatile silicones having from 2 to 9 silicon atoms and the viscosity of which is lower than or equal to 5×10⁻⁶ m²/s at 25° C., as for example decamethyl tetrasiloxane marketed, for example, under the trade name “SH 200” by the TORAY SILICONE company. Silicones belonging to this class are also described in the article TODD & BYERS, “Volatile Silicone fluids for cosmetics,” published in Cosmetics and Toiletries, Vol. 91, January 1976, P. 27-32.

According to at least one embodiment, non volatile silicones are used, for example polyalkyl siloxanes, polyaryl siloxanes, polyalkylaryl siloxanes, silicone-based gums and resins, polyorganosiloxanes modified with organofunctional groups, as well as mixtures thereof.

These silicones may be chosen from polyalkyl siloxanes, from which polydimethyl siloxanes with trimethylsilyl end groups may be mentioned, for example. Silicone viscosity is measured at 25° C. according to ASTM 445 standard, Appendix C.

These polyalkyl siloxanes encompass, as non-limiting examples, the following commercial products:
SILBIONE® oils of 47 and 70 047 series or MIRASIL® oils marketed by RHODIA, such as for example the oil 70 047 V 500 000;
oils of MIRASIL® series marketed by the RHODIA company;
oils of the 200 series from the DOW CORNING company, such as DC200 (viscosity 60 000 mm²/s);
VISCASIL® oils from GENERAL ELECTRIC and certain oils of the SF (SF 96, SF 18) series from GENERAL ELECTRIC.

Dimethylsilanol end group-containing polymethyl siloxanes, known under the name dimethiconol (CTFA) may also be mentioned, such as the oils of the 48 series from the RHODIA company.

This polyalkyl siloxane class also includes products marketed under the trade names “ABIL WAX® 9800 and 9801” by the GOLDSCHMIDT company, which are (C₁-C₂₀)polyalkyl siloxanes.

Polyalkylaryl siloxanes may be, in at least one embodiment, chosen from linear and/or branched polydimethyl/methylphenyl siloxanes, polydimethyl/diphenyl siloxanes having viscosities ranging from 1×10⁻⁵ to 5×10⁻² m²/s at 25° C.

Suitable examples of such polyalkylaryl siloxanes include, but are not limited to, products marketed under the following trade names:
SILBIONE® oils of the 70 641 series from RHODIA;
oils of RHODORSIL® 70 633 and 763 series from RHODIA;
DOW CORNING 556 COSMETIC GRAD FLUID from DOW CORNING;
silicones of the PK series from BAYER, such as the PK20 product;
silicones of the PN, PH series from BAYER, such as PN1000 and PH1000 products; and
certain oils of the SF series from GENERAL ELECTRIC, such as SF 1023, SF 1154, SF 1250, SF 1265.

As disclosed herein, suitable silicone gums include, but are not limited to, polyorganosiloxanes having high number average molecular weights ranging from 200,000 to 1,000,000 used either alone or in combination in a solvent. This solvent may be chosen from volatile silicones, polydimethyl siloxane (PDMS) oils, polyphenylmethyl siloxane (PPMS) oils, isoparaffins, polyisobutylenes, methylene chloride, pentane, dodecane, tridecane and mixtures thereof.

The following products may be mentioned, for example:

Polydimethyl siloxane;

Polydimethyl siloxane/methylvinyl siloxane gums;

Polydimethyl siloxane/diphenyl siloxane;

Polydimethyl siloxane/phenylmethyl siloxane; and

Polydimethyl siloxane/diphenyl siloxane/methylvinyl siloxane.

For instance, products that can be used according to the present disclosure include but are not limited to mixtures such as:

mixtures formed from an end chain-hydroxylated polydimethyl siloxane also called dimethiconol (CTFA) and a cyclic polydimethyl siloxane, also called cyclomethicone (CTFA), such as the Q2 1401 product marketed by the DOW CORNING company;

mixtures formed from a polydimethyl siloxane gum and a cyclic silicone, such as the SF 1214 Silicone Fluid from the GENERAL ELECTRIC company, this product being a SF 30 gum corresponding to a dimethicone, having a number average molecular weight of 500,000, solubilized in the SF 1202 Silicone Fluid corresponding to decamethyl cyclopentasiloxane;

mixtures from two PDMS with different viscosities, for instance from a PDMS gum and a PDMS oil, such as the SF 1236 product from GENERAL ELECTRIC. SF 1236 is a mixture from a SE 30 gum as defined above with a viscosity of 20 m²/s and a SF 96 oil with a viscosity of 5×10⁻⁶ m²/s. Such product comprises 15% of SE 30 gum and 85% of SF 96 oil, for example.

Organopolysiloxane resins that can be used according to the present disclosure include, but are not limited to, crosslinked siloxane systems comprising units:

R₂SiO_{2/2}, R₃SiO_{1/2}, RSiO_{3/2} and SiO_{4/2}, wherein R is chosen from a hydrocarbon group having from 1 to 16 carbon atoms and from a phenyl group. Amongst these products, those wherein R represents a lower C₁-C₄ alkyl group, such as a methyl group, or a phenyl group are used in at least one embodiment.

These resins also include the product marketed under the trade name “DOW CORNING 593” or those marketed under the trade names “SILICONE FLUID SS 4230 and SS 4267” by the GENERAL ELECTRIC company and which are dimethyl/trimethyl siloxane-structured silicones.

Resins of the trimethyl siloxysilicate type marketed, for example, under the trade names X22-4914, X21-5034 and X21-5037 by the SHIN-ETSU company may also be mentioned.

Organomodified silicones to be suitably used according to the present disclosure include, but are not limited to, silicones such as previously defined, and comprising in their structure at least one organofunctional group(s) bound through a hydrocarbon group.

Organomodified silicones to be suitably used according to the present disclosure include, but are not limited to, polyorganosiloxanes comprising:

polyethyleneoxy and/or polypropyleneoxy groups optionally comprising C₆-C₂₄ alkyl groups, such as products called dimethicone copolyol marketed by the DOW CORNING company under the trade name DC 1248 or SILWET® L 722, L 7500, L 77, L 711 oils from the UNION CARBIDE company and (C₁₂)alkyl methicone copolyol marketed by the DOW CORNING company under the trade name Q2 5200;

amine groups, substituted or not, such as the products marketed under the trade name GP 4 Silicone Fluid and GP 7100 by the GENESEE company, or the products marketed under the trade names Q2 8220 and DOW CORNING 929 or 939 by the DOW CORNING company. Substituted amine groups include, but are not limited to, C₁-C₄ aminoalkyl groups.

thiol groups, such as the products marketed under the trade names GP 72A et GP 71 from GENESEE.

alkoxyl groups, such as the product marketed under the trade name “SILICONE COPOLYMER F-755” by SWS SILICONES and ABIL WAX® 2428, 2434 and 2440 by the GOLDSCHMIDT company.

hydroxyl groups, such as the hydroxyalkyl function-containing polyorganoxiloxanes described in French Patent Application No. FR-A-85/16334;

alkoxyalkyl groups, such as for example the polyorganosiloxanes described in U.S. Pat. No. 4,957,732;

carboxylic type anionic groups such as, for example, in products described in European Patent No. EP 186 507 from the CHISSO CORPORATION company, or alkyl carboxylic type anionic groups such as those comprised in the X-22-3701E product from the SHIN-ETSU company; or 2-hydroxyalkyl sulfonate; 2-hydroxyalkyl thiosulfate such as the products marketed by the GOLDSCHMIDT company under the trade names ABIL® S201 and ABIL® S255;

hydroxyacrylamino groups, such as the polyorganosiloxanes described in European Patent Application No. EP 342 834. The Q2-8413 product from the DOW CORNING company is an example thereof.

The silicones such as described above may be used either alone or in combination, in a total amount ranging from 0.01 to 20% by weight, for example from 0.1 to 5% by weight, relative to the total weight of the composition.

The compositions of the present disclosure may also comprise fatty components such as mineral, vegetable, animal and synthetic oils, waxes, fatty esters, fatty alcohols, and fatty acids.

Suitable examples of oils that can be used in the composition of the present disclosure include but are not limited to:

animal-based hydrocarbon oils, such as perhydrosqualene;

vegetable-based hydrocarbon oils, such as liquid triglycerides of fatty acids comprising from 4 to 10 carbon atoms such as triglycerides of the heptanoic or octanoic acids, or for example sunflower oil, corn oil, soja bean oil, pumpkin oil, grape seed oil, sesame oil, nut oil, apricot kernel oil, macadamia nut oil, arara oil, castor oil, avocado oil, triglycerides of caprylic/capric acids such as those marketed by the Stearineries Dubois company or those sold under the names Miglyole® 810, 812 and 818 by the Dynamit Nobel company, jojoba oil, shea butter oil;

linear or branched, mineral or synthetic hydrocarbons, such as volatile or non volatile paraffin oils, and their derivatives, petrolatum, polydecenes, hydrogenated polyisobutene such as Parleam®; isoparaffines such as isohexadecane and isodecane; and

partly hydrocarbon-based and/or silicone-based fluorinated oils, such as those described in the Japanese Patent Application No. JP-A-2-295912; fluorinated oils also encompass perfluoromethyl cyclopentane and perfluoro-1,3 dimethylcyclohexane, sold under the names “FLUTEC® PC1” and “FLUTEC® PC3” by the BNFL Fluorochemicals company; perfluoro-1,2-dimethyl cyclobutane; perfluoroalkanes such as dodecafluoropentane and tetradecafluorohexane, sold under the names “PF 5050®” and “PF 5060®” by the 3M company, or bromoperfluorooctyle sold under the trade name “FORALKYL®” by the Atochem company; nonafluoromethoxybutane and nonafluoroethoxyisobutane; perfluoromorpholine derivatives, such as 4-trifluoromethyl perfluoromorpholine sold under the trade name “PF 5052®” by the 3M company;

The at least one wax can be chosen from carnauba wax, candellila wax, and alfa wax, paraffin, ozokerite, vegetable waxes such as olive tree wax, rice wax, hydrogenated jojoba wax or flower absolute waxes such as ribes nigrum (blackcurrant) flower wax sold by the BERTIN company (France), animal waxes such as beeswax, or modified beeswaxes (cerabellina); other waxes or wax-based raw materials to be used according to the present disclosure are also marine waxes such as the one sold by the SOPHIM company under the reference M82, polyethylene waxes or polyolefins in general.

Saturated or unsaturated fatty acids can be chosen from myristic acid, palmitic acid, stearic acid, behenic acid, oleic acid, linoleic acid, linolenic acid and isostearic acid, for example.

Fatty esters can be chosen from carboxylic acid esters, for instance mono, di, tri or tetracarboxylic esters.

As disclosed herein, the carboxylic acid esters can be saturated or unsaturated, linear or branched, C₁-C₂₆ aliphatic acid esters and saturated or unsaturated, linear or branched, C₁-C₂₆ aliphatic alcohol esters, where the total number of the ester carbon atoms is higher or equal to 10.

Suitable examples of monoesters to be mentioned include but are not limited to dihydroabietyl behenate; octyldodecyl behenate; isocetyl behenate; cetyl lactate; C₁₂-C₁₅ alkyl lactate, isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, ethyl-2-hexyl palmitate, 2-octyldecyl palmitate, alkyl myristates, such as isopropyl, butyl, cetyl, 2-octyldodecyl myristate, hexyl stearate, butyl stearate, isobutyl stearate; dioctyl malate, hexyl laurate, 2-hexyldecyl laurate.

C₄-C₂₂ di- or tricarboxylic acid and C₁-C₂₂ alcohol esters may also be used, as well as mono-, di- or tricarboxylic acid esters and di-, tri-, tetra- or pentahydroxy C₂-C₂₆ alcohol esters.

Non-limiting mention can be made of: diethyl sebacate; diisopropyl sebacate; diisopropyl adipate; di n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetraerygonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyidodecyl citrate; trioleyl citrate; propylene glycol dioctanoate; neopentyl glycol diheptanoate; diethylene glycol diisanonate; and polyethylene glycol distearates.

Among the previously mentioned esters, at least one embodiment of the present disclosure uses ethyl and isopropyl palmitates, ethyl-2-hexyl palmitate, 2-octyldecyl palmitate, alkyl myristates, such as isopropyl, butyl, cetyl, 2-octyldodecyl myristate, hexyl stearate, butyl stearate, isobutyl stearate; dioctyl malate, hexyl laurate, 2-hexyldecyl laurate and isononyl isononanate, cetyl octanoate.

Suitable fatty alcohols include, for example, saturated or unsaturated, linear or branched fatty alcohols having from 8 to 26 carbon atoms, such as cetyl alcohol, stearyl alcohol and mixture thereof (cetylstearyl alcohol), octyldodecanol, 2-butyloctanol, 2-hexyldecanol, 2-undecylpentadecanol, oleic alcohol and linoleic alcohol.

Fatty components may be present in an amount ranging from 0.1 to 50%, for instance from 1 to 30%, and even further, for example from 2 to 20% by weight, relative to the total weight of the composition.

The cosmetically acceptable medium of the composition, in addition to water, may further comprise at least one organic solvent.

The at least one organic solvent, in at least one embodiment, is chosen from C₁-C₆ alcohols, for example alkanols, such as ethanol, propanol and isopropanol, alkanediols such as propylene glycol and pentanediols, benzyl alcohol, C₅-C₁₀ alkanes, acetone, methyl ethylcetone, methyl acetate, butyl acetate, ethyl acetate, dimethoxyethane, diethoxyethane and mixtures thereof.

The at least one organic solvent may be present in an amount ranging from 0.5 to 80% and further, for example, from 1 to 50% by weight, relative to the total weight of the composition.

The person skilled in the art will be able to add some additives without affecting the beneficial properties of the compositions of the present disclosure.

The compositions of the present disclosure may be in the form of hairstyling compositions which may be rinsed off, such as styling shampoos or not rinsed off, such as styling lotions, foams or gels. For instance, they may be in the form of styling lotions or gels.

They also may be in the form of an aerosol. In that case, the composition will further comprise a propellant. As is understood by one of ordinary skill, said propellant may be a gas or a mixture of compressed or liquefied gases, that may optionally be dissolved in the composition. Non-limiting examples of suitable gas propellants encompass air, carbon dioxide, nitrogen, dimethyl ether, hydrocarbons such as propane, n-butane, isobutane or isopentane and halogenated hydrocarbons, for example fluorinated hydrocarbons, and mixtures thereof.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in its respective testing measurement.

The examples that follow are intended to illustrate the present disclosure without, however, being limiting in nature.

### EXAMPLES

### Formulation Examples

### Example 1

### Styling Lotion

| | | |
|---|---|---|
| Cationic polyurethane containing a polyolefin | 3% | a.m. |
| sequence¹ | | |
| Copolymer of vinyl pyrrolidone/vinyl acetate² | 3% | a.m. |
| JAGUAR HP 105 (RHODIA)³ | 1.5% | a.m. |

| | |
|---|---|
| Demineralized water | Qs 100% |

### Example 2

### Styling Gel

| | | |
|---|---|---|
| Cationic polyurethane containing a polyolefin | 3% | a.m. |
| sequence¹ | | |
| Copolymer of vinyl pyrrolidone/vinyl acetate² | 3% | a.m. |
| Polyethylene glycol distearate⁴ | 3% | a.m. |
| Oxyethylene glyceryl monostearate⁵ | 2% | a.m. |
| Propylene glycol | 2.5% | a.m. |
| JAGUAR HP 105 (RHODIA)³ | 1% | a.m. |

| | |
|---|---|
| Demineralized water | Qs 100% |

¹(A) Polyurethane in an aqueous dispersion formed from 8.7% of N-methyl diethanol amine, 23.4% of isophorone diisocyanate, 67.9% of KRASOL LBH2000 (polybutadiene with hydroxyl end functions), and neutralized up to 40% using hydrogen chloride.
²PVP/VA S 630 (ISP)
³Gelling agent (hydroxypropyl guar)
⁴Polyethylene glycol 6000 distearate (AKZO NOBEL)
⁵SIMULSOL ® 220 TM (SEPPIC)

The compositions described in the examples above were prepared, and good cosmetic properties were obtained, together with a strong and very long-lasting hair fixation.

A similar result was obtained with a (B) polyurethane in an aqueous dispersion formed from 8.4% of poly(tetramethylene oxide), 8.6% of N-methyl diethanol amine, 21.4% of isophorone diisocyanate, 61.6% of KRATON L2203 (polybutadiene with hydroxyl end functions), and neutralized up to 40% using hydrogen chloride.

What is claimed is:

1. A cosmetic composition comprising, in a cosmetically acceptable aqueous medium:
(i) at least one cationic polyurethane comprising at least one non ionic unit derived from an olefinic homopolymer and/or copolymer, and
(ii) at least one vinyl pyrrolidone homopolymer or copolymer.

2. The cosmetic composition according to claim 1, wherein at least 50% by weight of the polyurethane non ionic units are derived from an olefinic homopolymer or copolymer.

3. The cosmetic composition according to claim 1, wherein all the polyurethane non ionic units are derived from an olefinic homopolymer and/or copolymer.

4. The cosmetic composition according to claim 1, wherein said olefinic homopolymers and copolymers are homopolymers and copolymers carrying labile hydrogen functions at their ends, and comprising units chosen from ethylene, propylene, 1-butylene, 2-butylene, isobutylene, 1,2-butadiene, 1,4-butadiene, and isoprene units and mixtures thereof.

5. The cosmetic composition according to claim 4, wherein said olefinic homopolymers and copolymers are derived from optionally hydrogenated 1,2- and/or 1,4-butadiene.

6. The cosmetic composition according to claim 1, wherein the at least one cationic polyurethane comprises:
(a) cationic units resulting from the reaction of at least one tertiary or quaternary amine comprising at least two labile hydrogen-containing reactive functions;
(b) non ionic units, wherein at least one unit (b1) of which results from the reaction of at least one polymer chosen from olefinic homopolymers and copolymers carrying labile hydrogen-containing reactive functions at their ends and having a glass transition temperature (Tg) lower than 10° C.; and
(c) units resulting from the reaction of at least one diisocyanate.

7. The cosmetic composition according to claim 6, wherein the cationic (a) units result from the reaction of at least one tertiary or quaternary amine comprising two labile hydrogen-containing reactive functions.

8. The cosmetic composition according to claim 7, wherein said at least tertiary or quaternary amine is chosen from amines having following formulas: 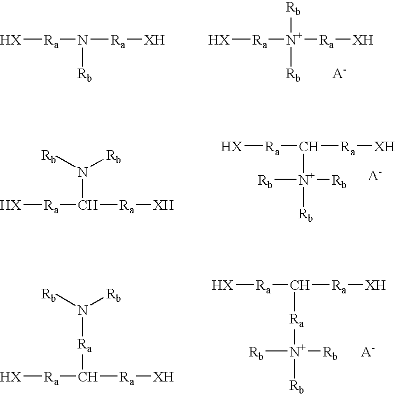
wherein:
each Rₐ is independently chosen from linear or branched C₁-C₆ alkylene groups, C₃-C₆ cycloalkylene groups, arylene groups, and mixtures thereof; wherein all of them may be optionally substituted with at least one halogen atom and optionally comprise at least one heteroatom chosen from O, N, P and S;
each R_{b} is independently chosen from C₁-C₆ alkyl groups, C₃-C₆ cycloalkyl groups, aryl groups, and mixtures thereof; wherein all of them may be substituted with at least one halogen atom and comprise at least one halogen atom and comprise at least one heteroatom chosen from O, N, P and S;
each X is independently chosen from oxygen and sulfur atoms and from NH and NR_{c} groups, wherein R_{c} is a C₁-C₆ alkyl group; and
A⁻ is a physiologically acceptable counter-ion.

9. The cosmetic composition according to claim 8, wherein the cationic (a) units result from the reaction of N-methyldiethanol amine or N-tert-butyldiethanol amine.

10. The cosmetic composition according to claim 7, wherein the (a) units result from the reaction of at least one tertiary and/or quaternary amine function-containing polymer, carrying labile hydrogen-containing reactive functions at their ends chosen from —OH, —NH₂, —NHR_{c} or —SH, and having a weight average molecular weight ranging from 400 to 10,000, wherein R_{c} is a C₁-C₆ alkyl group.

11. The cosmetic composition according to claim 6, wherein the cationic polyurethane optionally comprises at least one non ionic (b2) unit, different from the (b1) unit, derived from a non ionic monomer compound comprising at least two labile hydrogen functions that can react with said (c) compounds comprising at least one diisocyanate.

12. The cosmetic composition according to claim 11,
wherein the cationic (a) units are present in an amount ranging from 0.1 to 90% by weight of the cationic polyurethane total units,
wherein the non ionic units derived from a (b1) olefinic homo- or copolymer are present in an amount ranging from 10 to 99.9% by weight of the cationic polyurethane total units, and
wherein the (b2) non ionic units are present in an amount ranging from 0 to 50% by weight of the cationic polyurethane total units.

13. The cosmetic composition according to claim 6, wherein the at least one diisocyanate is chosen from methylenediphenyl diisocyanate, methylenecyclohexane diisocyanate, isophorone diisocyanate, toluene diisocyanate, naphthalene diisocyanate, 1,4-butane diisocyanate, and 1,6-hexane diisocyanate.

14. The cosmetic composition according to claim 13, wherein the at least one diisocyanate is isophorone diisocyanate.

15. The cosmetic composition according to claim 6, wherein the (c) units are present in an amount ranging from 1 to 60% by weight of the cationic polyurethane total units.

16. The cosmetic composition according to claim 15, wherein the (c) units are present in an amount ranging from 1 to 40% by weight of the cationic polyurethane total units.

17. The cosmetic composition according to claim 6, wherein the non ionic monomer compound(s) forming the (b2) non ionic units are chosen from C₁-C₁₂ diols, preferably neopentyl glycol, hexa(ethylene glycol), 1,2-ethanediol, 1,2-propanediol and 1,3-propanediol, and C₁-C₆ aminoalcohols.

18. The cosmetic composition according to claim 17, wherein said non ionic monomer compound(s) forming the (b2) non ionic units are aminoethanol.

19. The cosmetic composition according to claim 6, wherein the cationic polyurethane does not comprise any further unit in addition to (a), (b) and (c) units.

20. The cosmetic composition according to claim 6, wherein the cationic polyurethane is elastic.

21. The composition according to claim 1, wherein the at least one cationic polyurethane is present in an amount ranging from 0.01% to 40%, by weight, relative to the total weight of the cosmetic composition.

22. The composition according to claim 21, wherein the at least one cationic polyurethane is present in an amount ranging from 0.1% to 10%, by weight, relative to the total weight of the cosmetic composition.

23. The composition according to claim 1, wherein said vinyl pyrrolidone homo- and/or copolymers are chosen from polyvinyl pyrrolidones (PVP), polyvinyl pyrrolidones complexed with iodine, copolymers of polyvinyl pyrrolidone/vinyl acetate (PVP/VA), polyvinyl pyrrolidone/butene; polyvinyl pyrrolidone/decene, polyvinyl pyrrolidone/hexadecene, polyvinyl pyrrolidone/dimethyl aminoethyl methacrylate (PVP/DMAEMA), polyvinyl pyrrolidone/dimethylaminopropyl(meth)acrylamide, polyvinyl pyrrolidone/(meth)acrylic acid/lauryl methacrylate, polyvinyl pyrrolidone/vinyl acetate/itaconic acid, polyvinyl pyrrolidone/vinyl acetate/vinyl propionate and polyvinyl pyrrolidone/vinyl caprolactame/dimethylaminopropyl acrylamide/acrylic acid or ester.

24. The composition according to claim 1, wherein said vinyl pyrrolidone homo- and/or copolymer is chosen from polyvinyl pyrrolidones and copolymers of vinyl pyrrolidone/vinyl acetate.

25. The composition according to claim 1, wherein said at least one homo- or copolymer of vinyl pyrrolidone is present in an amount ranging from 0.01 to 20% by weight, relative to the total weight of the cosmetic composition.

26. The composition according to claim 25, wherein said at least one homo- or copolymer of vinyl pyrrolidone is present in an amount ranging from 0.5% to 5% by weight, relative to the total weight of the cosmetic composition.

27. The composition according to claim 1, further comprising at least one additive chosen from gelling agents and/or thickeners, surfactants, silicones, organic solvents, fragrances, mineral, vegetable and/or synthetic oils, fatty acid esters, pH stabilizing agents, preserving agents and UV absorbers.

28. The composition according to claim 1, further comprising at least one propellant, wherein the composition is in the form of an aerosol.

29. A hairstyling method comprising:
applying onto the hair a composition wherein the composition comprises, in a cosmetically acceptable aqueous medium:
(i) at least one cationic polyurethane comprising at least one non ionic unit derived from an olefinic homopolymer and/or copolymer, and
(ii) at least one vinyl pyrrolidone homopolymer or copolymer, and
then optionally rinsing the hair, and optionally styling and drying the hair.
